(19) United States
(12) Patent Application Publication
(10) Pub. No.: US 2008/0318283 A1
(43) Pub. Date: Dec. 25, 2008

(54) Fermentation Process for Continuous Plasmid Dna Production

(75) Inventor: Aaron E. Carnes, Lincoln, NE (US)

Correspondence Address: BROOKS KUSHMAN P.C., 1000 TOWN CENTER, TWENTY-SECOND FLOOR, SOUTHFIELD, MI, 48075, US

(73) Assignees: Clague P. Hodgson, Omaha, NE (US); NATURE TECHNOLOGY CORPORATION, Lincoln, NE (US)

(21) Appl. No.: 12/162,689

(22) PCT Filed: Jan. 31, 2007
(86) PCT No.: PCT/US07/02707

§ 371 Date: Jul. 30, 2008

Related U.S. Application Data
(60) Provisional application No. 60/764,042, filed on Feb. 1, 2006.

(51) Int. Cl. C12N 15/64

(52) U.S. Cl. 435/91.4

(57) ABSTRACT

A continuous process is described for the production of microbial plasmid DNA for use in biopharmaceutical and biotechnological applications. The process consists of: first growing microbial cells containing a plasmid at a reduced temperature in a continuous stage; followed by a second plasmid induction continuous culture stage with an increased temperature, with a residence time that allows accumulation of the plasmid product. A hold step at a reduced temperature after fermentation further increases the yield of plasmid product. The method enables production of a large quantity of highly purified plasmid DNA from a small bioreactor over time.

### CROSS REFERENCE TO RELATED APPLICATIONS

This application is entitled to the benefit of Provisional Patent Application Ser. No. US60/764,042 filed 1 Feb. 2006

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND

1. Field of Invention

The present invention relates to the production of covalently closed circular (ccc) recombinant DNA molecules such as plasmids, cosmids, bacterial artificial chromosomes (BACs), bacteriophages, viral vectors and hybrids thereof.

2. Description of Prior Art

E. coli plasmids have long been the single most important source of recombinant DNA molecules used by researchers and by industry. Today, plasmid DNA is becoming increasingly important as the next generation of biotechnology products (gene medicines and DNA vaccines) make their way into clinical trials, and eventually into the pharmaceutical marketplace. Plasmid DNA vaccines may find application as preventive vaccines for viral, bacterial, or parasitic diseases; immunizing agents for the preparation of hyper immune globulin products; therapeutic vaccines for infectious diseases; or as cancer vaccines. Plasmids are also utilized in gene therapy or gene replacement applications, wherein the desired gene product is expressed from the plasmid after administration to the patient.

Today, the FDA standards are not defined except in preliminary form (see: FDA Points to Consider on Plasmid DNA Vaccines for Preventive Infectious Disease Indications, 1996). However, in the future, international standards for plasmid DNA purity are likely to be the same or very similar to those that are used for recombinant protein products similarly produced from E. coli fermentation, and such standards exceed the current purity attainable from established methods. Most glaringly, the accepted standard of <100 pg host genomic DNA per dose (see: FDA Points to consider in the characterization of cell lines used to produce biologics, 1993) is far below the levels currently attainable for purified plasmid preparations (100 pg per 1 mg dose is equivalent to one part per ten million).

The basic methods for obtaining plasmids (by bacterial fermentation), and for their purification (e.g., by the alkaline lysis method) are well-known (Birnboim, & Doly, Nucleic Acids Res. 7:1513-1523 (1979)). Initially, the fermented bacterial cell paste is resuspended and lysed (using a combination of sodium hydroxide and sodium dodecylsulfate), after which the solution is neutralized by the addition of acidic salt (e.g., potassium acetate), which precipitates the bacterial DNA and the majority of cell debris. The bulk of super-coiled plasmid DNA remains in solution, along with contaminating bacterial RNA, DNA and proteins, as well as E. coli endotoxin (lipopolysaccharide, or LPS). The soluble fraction is then separated by filtration and subjected to a variety of purification steps, which may include: RNase digestion; chromatography (ion exchange gel filtration, hydroxyapatite, gel filtration, hydrophobic interaction, reverse phase, HPLC, etc.); diafiltration; organic extraction, selective precipitation, etc.

Clearly, increasing the purity of the starting material and achieving better downstream purification are essential goals for manufacturing clinical grade DNA on an industrial scale.

### Fermentation Media Considerations

Design of a balanced medium is based on the cell's energy requirements and elemental composition. Typically, the nutritional requirements are satisfied by either minimal media or semi-defined media.

Semi-defined media contain complex components such as yeast extract, casamino acids, and peptones. The addition of complex components supplies growth factors, amino acids, purines and pyrimidines and often supports higher cell densities.

A carbon source is commonly included in the highest amounts. The carbon source provides energy and biomass, and is usually utilized as the limiting nutrient. Glucose is the conventional carbon source. It is metabolized very efficiently and therefore gives a higher cellular yield. However, high glucose concentrations cause undesirable acetate production due to metabolic overflow (known as the Crabtree effect). Glycerol is also used and is often the preferred carbon source in batch cultures. Although cellular yields from glycerol are slightly smaller than from glucose, glycerol does not cause as high of levels of acetate production and can be used at higher concentrations without being inhibitory. Glycerol also supports reduced maximum specific growth rates.

The requirement for nitrogen may be satisfied by inorganic or organic nitrogen sources. Ammonia and ammonium salts (e.g. NH₄Cl, (NH₄)₂SO₄) are used in minimal media. Semi defined media supply nitrogen either partly or entirely from complex components, including yeast extract, peptones, and casamino acids.

Minerals are required for growth, metabolism, and enzymatic reactions. Magnesium, phosphorus, potassium, and sulfur are typically added as distinct media components. Di- and monopotassium phosphates provide potassium and phosphorous and also function as buffering agents in certain proportions. Magnesium sulfate heptahydrate is often used as the source of magnesium and sulfur. Other essential minerals include calcium, copper, cobalt, iron, manganese, molybdenum and zinc. These are required in smaller amounts and are often supplied by addition of a trace minerals solution, though they are usually present as impurities in the major ingredients. Osmolarity is adjusted with sodium chloride.

The use of animal-derived products, and in particular bovine products, in plasmid manufacture is unacceptable due to the risk of prion or virus contamination. All media components should be certified animal product free. Vegetable-derived substitutes are available for many components which have animal origin (e.g. vegetable glycerol, soy peptone).

### Plasmid Fermentation Process Considerations

### Growth Rate

The use of reduced growth rate is the unifying principle in high quality, high yield plasmid fermentations. High growth rates have been associated with acetate production, plasmid instability, and lower percentages of super-coiled plasmid. A reduced growth rate alleviates growth rate-dependent plasmid instability by providing time for plasmid replication to synchronize with cell division.

### Growth Conditions

Cultivation in a bioreactor gives us the ability to control and monitor many of the parameters that affect plasmid quality and yield. Super-coiling is known to be affected by oxygen and temperature (Dorman et al., J. Bact. 179:2816-2826 (1988), Goldstein & Drlica Proc. Natl. Acad. Sci. USA. 81: 4046-4050, (1984)) Oxygen has been shown to play a role in plasmid stability. One study (Hopkins et al., Biotechnol. Bioeng. 29: 85-91, (1987)), found that a single drop in dissolved oxygen concentration to 5% of air saturation led to rapid loss in plasmid stability. Another study (Namdev et al., Biotechnol. Bioeng. 41: 666-670, (1993)) showed that fluctuations in oxygen input lead to plasmid instability. Furthermore, the formation of nicked plasmids and multimers can be affected by many parameters, including temperature, pH, dissolved oxygen, nutrient concentration, and growth rate (Durland & Eastman, Adv. Drug Deliver. Rev. 30:33-48, 1998)). The optimal temperature for E. coli growth is 37° C. However, lower temperatures (30-37° C.) may be used in batch fermentation to cause a reduced maximum specific growth rate. Higher temperature (42° C.) can also be employed to induce selective plasmid amplification with some replication origins such as pUC, and pMM1 (Wong et al., Proc. Natl. Acad. Sci. USA. 79: 3570-3574, (1982), Lin-Chao et al., Mol. Microbiol. 6: 3385-3393, (1992)) and runaway replicon R plasmids. Hamann et. al. World Patent Application WO0028048, (2000) report a process for the production of R plasmids wherein plasmid production is maintained at a low level (by use of low temperature) to avoid retardation of growth due to plasmid DNA synthesis; once the host cell population is high, plasmid production is induced by temperature shifting.

### Batch Fermentation

Batch fermentation has the main advantage of simplicity. All nutrients that will be utilized for cell growth and plasmid production throughout the culture period are present at the time of inoculation. Batch fermentation has a lag phase, exponential growth phase, and stationary phase. The use of a suitable inoculum (1-5% of the culture volume) will reduce the length of the lag phase. During the exponential phase all nutrients are in excess; thus the specific growth rate will be essentially the maximum specific growth rate, μₘₐₓ, as predicted by Monod kinetics. As discussed previously, reduced growth rates are desirable for plasmid production. In batch fermentation the growth rate can only be reduced by reducing μₘₐₓ. This has been achieved by growth at lower temperatures and by growth on glycerol instead of glucose. Batch fermentation at 30° C. using glycerol will typically result in μₘₐₓ≦0.3 h⁻¹, which is sufficient to prevent deleterious acetate accumulation and growth rate associated plasmid instability (Thatcher et al., U.S. Pat. No. 6,503,738). Glycerol can also be used at much higher concentrations than glucose without being inhibitory, leading to higher biomass yields. Generally, biomass yields of up to 60 g/L DCW can be obtained with batch fermentation.

### Fed-Batch Fermentation

Fed-batch fermentation is especially useful for plasmid production. Controlled addition of a limiting nutrient allows for control of growth rate at rates <μₘₐₓ. Also, fed-batch fermentation results in higher yields. The key to fed-batch fermentation is supplying substrate at a rate such that it is completely consumed. As a result, residual substrate concentration is approximately zero and maximum conversion of substrate is obtained. Metabolic overflow from excess substrate is avoided, reducing the formation of inhibitory acetate.

Fed-batch fermentation starts with a batch phase. Cells are inoculated into an initial volume of medium that contains all non-limiting nutrients and an initial concentration of the limiting substrate. Controlled feeding of the limiting nutrient begins once the cells have consumed the initial amount of substrate.

One of the simplest and most effective feeding strategies is exponential feeding. This method allows the culture to grow at a predetermined rate less than μₘₐₓ without the need of feedback control. The fermentation begins with a batch mode containing a non-inhibitory concentration of substrate. The cells grow at μₘₐₓ until the substrate is exhausted, at which point the nutrient feeding begins.

The DO-stat and pH-stat methods are fairly easy to implement since most standard fermentor systems include dissolved oxygen and pH monitoring. Trends in dissolved oxygen (DO) and pH can indicate whether substrate is available to the cells. Exhaustion of substrate causes decreased oxygen uptake and the DO concentration in the medium rises. The pH also rises due to consumption of metabolic acids. Feeding is triggered when DO or pH rises above a set threshold. The growth rate can be adjusted by changing the DO or pH threshold value.

### Continuous Fermentation

Continuous culture may be desirable for manufacturing very large quantities of plasmid. More plasmid can be produced by increasing productivity, rather than increasing the volume or number of fermentations. For example, a typical batch fermentation might last 20 hours and yield 30 g/L DCW. A continuous culture at steady state with a cell concentration of 30 g/L DCW and a dilution rate of 0.2 h⁻¹ can produce four times the amount of cell mass in 20 hours at a reduced μ. Continuous culture is most commonly performed as a chemostat. Medium added to the vessel at a rate F displaces an equal culture volume with cell concentration X through an overflow device. For example, in a chemostat the addition of nutrient to the vessel is stated in terms of dilution rate, D, defined as:

D=F/V

where V is the culture volume. The net change in cell concentration over time, dX/dt, equals growth minus output:

dX/dt=μX−DX

At steady state, cell concentration remains constant, so dX/dt=0, and:

μX=DX

μ=D

Therefore, under steady state conditions the specific growth rate may be controlled by the dilution rate at rates less than μₘₐₓ. The chemostat also provides a constant environment that may be optimized for plasmid replication.

Continuous culture has been previously used to investigate plasmid stability and maintenance in E. coli. While some plasmids have been shown to be stable after extended continuous culture (e.g. pDS1109, Jones et al., Mol. Gen. Genet. 180:579-584, (1980)), other common plasmids (e.g. pBR322, pMB9) were lost from their host cells during continuous culture (Jones et al., Supra). After an initial population of plasmid-free segregants appears, it will usually take over the culture because of lower metabolic burden, unless the plasmid gives its host cell a growth advantage over plasmid-free cells. In other experiments the plasmid was not lost from the host, but the copy-number was reported to drop in response to nutrient limitation (Jones et al., Supra). The stability of a plasmid in continuous culture has also been shown to be affected by the dilution rate, D; loss of plasmid from a culture occurs more rapidly at lower dilution rates and slows with increasing dilution rates (Wouters et al., Antonie van Leeuwenhoek 46:353-362, (1980)). Thus, continuous culture has not been previously utilized for manufacturing plasmid DNA.

### Exemplary Plasmid Fermentation Processes

Examination of current yields reveals that typical laboratory shake flask culture produces from 1-5 mg of plasmid DNA/L of culture, whereas a fermentor can produce, typically, from 10-250 mg/L.

Lahijani et al. (Human Gene Ther. 7: 1971-1980, (1996)) have reported using a pBR322-derived plasmid with a temperature sensitive single point mutation (pUC origin) in fermentation with exponential feeding and a temperature shift from 37° C. to 42-45° C. They achieved a plasmid yield of 220 mg/L in a 10 L fermentor. The same plasmid without the mutation in batch fermentation (pBR322 derived origin) at 30° C. yielded only 3 mg/L plasmid.

Friehs et al., U.S. Pat. No. 6,664,078, describe a fed-batch process using a glycerol yeast extract medium with DO-stat feedback controlled feeding. The fermentation started with an initial batch volume of 7.5 L. Agitation was increased to keep DO above 30%. Feed medium was pumped in when DO reached a threshold set point of 45%. The culture reached stationary phase after 41 hours, yielding 60 g/L DCW and 230 mg/L of plasmid.

Chen et al., U.S. Pat. No. 5,955,323, have used a fed-batch process in semi-defined medium with combination DO-stat and pH-stat feedback control. DO and pH threshold setpoints were 50% and 7.2, respectively. When DO dropped below 30% because of high metabolic activity agitation speed was increased by a percentage of the previous speed. In a 7 L fermentor, this strategy led to a specific growth rate of 0.13 h⁻¹ and plasmid yields of 82-98 mg/L.

Durland and Eastman, Supra, report batch fermentation at 37° C. in a proprietary medium. Their process typically yields 130 mg/L and has yielded as high as 250 mg/L.

Carnes et al. (Biotechnol. Appl. Biochem. 45:155-66, (2006), also international patent publications PCT/US2005/29238 and WO2006/023546, (2004)) describe a fed-batch fermentation process in which plasmid-containing E. coli cells are grown at a reduced temperature during the fed-batch phase, during which growth rate was also restricted. This was followed by a temperature up-shift and continued growth at elevated temperature to accumulate. An exponential feeding strategy was used to restrict the specific growth rate to approximately 0.12 h⁻¹. In this process, the specific plasmid yield continues to rise for up to 15 hours after the temperature up-shift. This process has led to plasmid yields as high as 2.1 g/L.

### Current Obstacles

The fermentation media and processes described above incorporate what is currently known in the art to improve plasmid productivity, such as reduced growth rate and pUC plasmid copy number induction with high temperature. Most of these processes plateau at about 200-250 mg plasmid/L. This low yield imposes a cost and purity burden on commercialization of plasmid DNA production processes. Although economies of scale will reduce the cost of DNA significantly in the future, a far more economical solution to this problem is needed in order to achieve the desired cost. As well, international standards for plasmid DNA purity are likely to be the same or very similar to those that are used for recombinant protein products similarly produced from E. coli fermentation, and such standards exceed the current purity attainable from established methods. Increasing the yield (mg of DNA/gram of cell paste) in fermentation would both decrease the cost and increase the purity of the DNA (because it reduces the amount of material being processed).

### BRIEF SUMMARY OF THE INVENTION

The invention is a multi-stage continuous fermentation processes, useful for plasmid DNA production.

### OBJECTS AND ADVANTAGES

One object and/or advantage of the invention is to improve plasmid DNA productivity from fermentation culture

Another object and/or advantage object of the invention is to improve plasmid DNA specific yield in fermentation culture

Another object and/or advantage of the invention is to improve plasmid DNA volumetric yield in fermentation culture.

Another object and/or advantage of the invention is to improve plasmid DNA quality in fermentation culture.

Another object and/or advantage of the invention is to increase the production of plasmid DNA with limited bioreactor volume, with respect to the amount of plasmid that can be produced using batch or fed-bath methods.

Another object and/or advantage of the invention is to reduce impurities in purified plasmid DNA.

We disclose improved fermentation processes that, compared to processes defined in the art are improved by:
1) Improved plasmid stability, yield, and integrity of plasmids from continuous culture by maintenance of low, more stable, plasmid levels during biomass production in the first stage vessel.
2) Increased yield of plasmid through induction of plasmid amplification to accumulate high plasmid levels in the second stage vessel.
3) Improved plasmid yield and plasmid quality through optional additional holding stages at reduced temperatures.
4) Improved plasmid productivity from fermentation by using continuous culture.
5) Increased quality of plasmid by reduced levels of nicked (open circular) or linearized versions of the plasmid.
6) Increased quality of plasmid by increasing the percent monomer of plasmid.
7) Simplified production using robust, automated control parameters and feeds.
8) Simplified scaling due to growth control and reduced oxygen demand during growth.
9) Reduced levels of impurities after plasmid purification due to enriched levels of plasmid in the feed stream into downstream processing.
10) Improved regulatory compliance by elimination of all animal product derived components.
   Further objects and/or advantages of the invention will become apparent from a consideration of the drawings and ensuing description.

### BRIEF DESCRIPTION OF THE SEVERAL VIEWS OF THE DRAWING FIGURES

FIG. 1. gWiz GFP inducible fed-batch fermentation in E. coli

FIG. 2. Two stage continuous process described in Example 2.

FIG. 3. Agarose gel electrophoresis of plasmid samples from the two stage continuous process described in Example 2.

FIG. 4. Two stage chemostat continuous fermentation process.

FIG. 5. Biomass and substrate concentrations for Example 3 in Vessel 1 and Vessel 2.

FIG. 6. Inducible fed-batch fermentation process

Table 1 Replication origins

Table 2 Host strains

### FULL DESCRIPTION OF THE DRAWING FIGURES

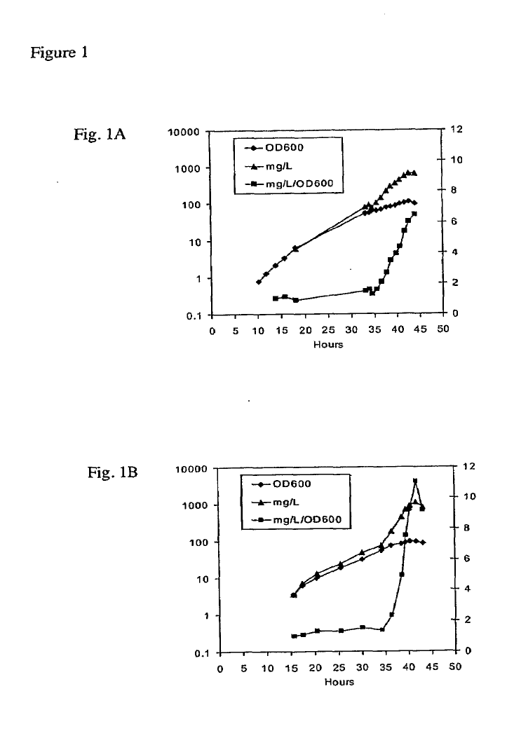

FIG. 1. gWiz GFP inducible fed-batch fermentation in E. coli with NTC3019 medium (37° C. or 42° C. induction) A) gWiz-GFP/DH5α fermentation with a 30→37° C. temperature shift at 35 hours. Plasmid yield reached 670 mg/L; B) gWiz-GFP/DH5α fermentation with a 30→42° C. temperature shift at 35 hours. Plasmid yield reached 1,100 mg/L.

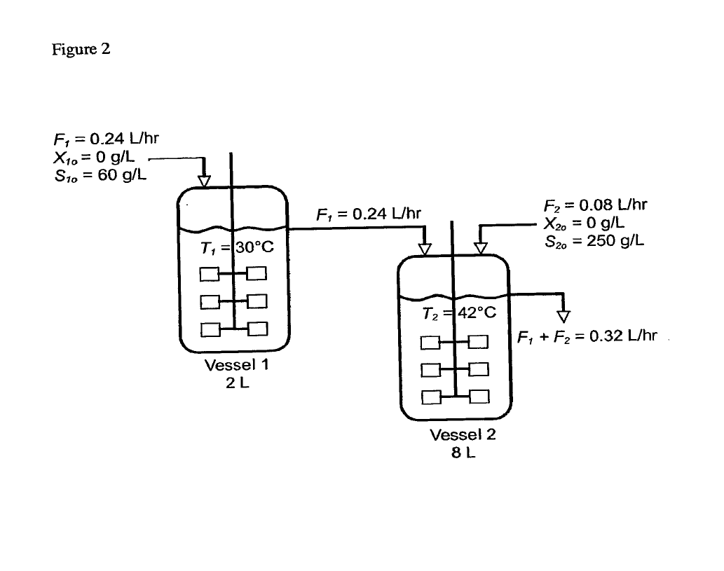

FIG. 2. Two stage continuous process described in Example 2.

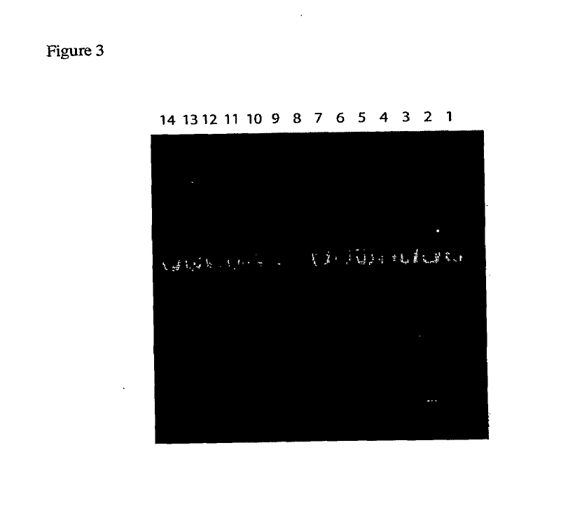

FIG. 3. Agarose gel electrophoresis showing stability of plasmid DNA isolated from samples taken from the two stage continuous process described in Example 2. Lanes 1-7: samples from the first stage; Lanes 8-14: samples from the second stage.

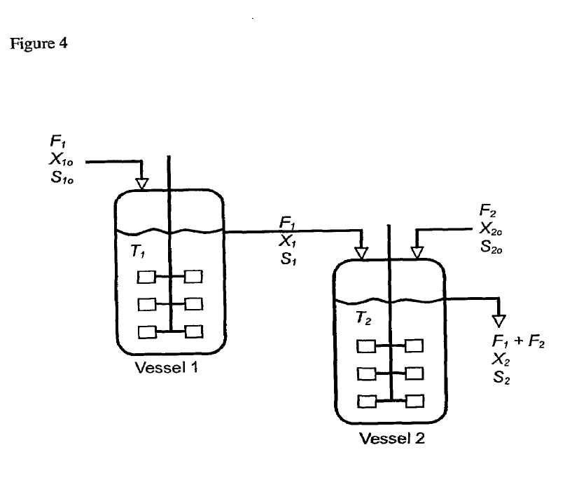

FIG. 4. Two stage chemostat continuous fermentation process. F₁, X₁ₒ, and S₁ₒ are volumetric feed rate, biomass concentration, and limiting substrate concentration, respectively, entering Vessel 1. X₁ and S₁ are the biomass and limiting substrate concentrations in and exiting Vessel 1. F₂, X₂ₒ, and S₂ₒ are volumetric feed rate, biomass concentration, and limiting substrate concentration, respectively, entering Vessel 2 from a separate feed. X₂ and S₂ are the biomass and limiting substrate concentrations in and exiting Vessel 2. T₁ and T₂ are the culture temperatures in Vessel 1 and Vessel 2.

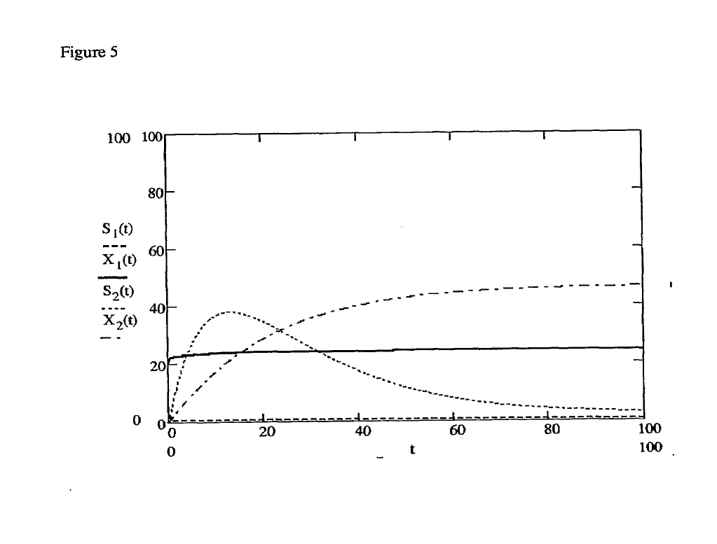

FIG. 5. Biomass and substrate concentrations for Example 3 in Vessel 1 and Vessel 2.

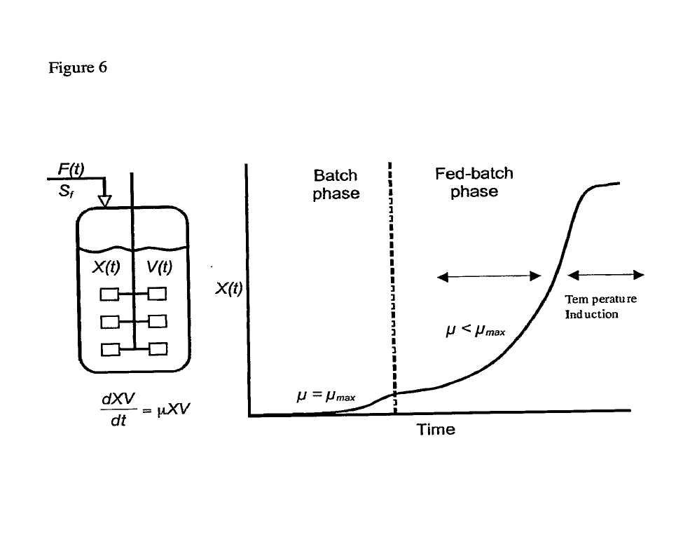

FIG. 6. Inducible fed-batch fermentation process

### DETAILED DESCRIPTION OF THE INVENTION

### Definitions

ccc: Covalently Closed Circular

ColE1 derived origin: Origin of replicated derived from ColE1 type plasmid (e.g. pMB1, ColE1) by deletion (e.g. pBR322 derived origin) and/or base change (e.g. pUC from pMB1, pMM1, pMM5 from ColE1 etc)

Continuous culture: Culture continuously supplied with nutrients by the inflow of fresh medium, with continuous outflow of culture.

NTC3018 fermentation media: Glycerol semi defined batch fermentation media

NTC3019 fermentation media: Glycerol semi defined fed-batch fermentation media

pDNA: Plasmid DNA

pBR322-derived origin: pMB1 origin from pBR322, in which the rop (repressor of primer) gene has been deleted

plasmid: plasmids, cosmids, bacterial artificial chromosomes (BACs), bacteriophages, viral vectors and hybrids thereof

pUC origin: pBR322-derived origin, with G to A transition that increases copy number at elevated temperature

semi-defined glycerol media: fermentation media that contains complex nitrogen source (e.g. yeast extract, soy extract) and glycerol carbon source

### DETAILED DESCRIPTION OF THE INVENTION

The invention is practiced in the production of covalently closed circular (ccc) recombinant DNA molecules such as plasmids, cosmids, bacterial artificial chromosomes (BACs), bacteriophages, viral vectors and hybrids thereof (herein collectively referred to as plasmids).

Fermentation processes described in the art are not optimal, with suboptimal plasmid yield, quality (e.g. nicking or linearization of plasmid), poor scalability (e.g. due to excessive oxygen supplementation requirements), and restricted application (e.g. inability to use with plasmids containing unstable or toxic sequences).

### Continuous Plasmid Production Process and Media Preferred Embodiments

A novel multistage continuous culture process for plasmid production is disclosed herein. This features a first stage for biomass production combined with a second stage with novel feeding strategies and conditions designed for increased plasmid amplification. We also contemplate additional stages for stabilizing the culture prior to harvest.

The discovery that a multistage continuous culture process results in increased plasmid yield and productivity is not taught in the art. Wouters et al., Antonie von Leeuwenhoek 46:353-362 (1980) found that in continuous cultures of E. coli containing plasmid DNA, the plasmid was often unstable; this happened most severely at higher temperatures and reduced growth rates. Although reduced temperatures may be used to improve plasmid stability in continuous culture, Lahijani et al., Supra, showed that plasmid copy number is reduced at lower temperatures, and the overall goal of improved plasmid yield is not met. Lahijani et al. Supra, did show that plasmid yield may be greatly increased by using higher temperatures, but as Wouters et al., Supra, demonstrated, extended cultivation at these temperatures results in plasmid instability.

The temperature inducible fed-batch process of Carnes et al, 2004 & 2006, Supra, utilizes restricted growth and temperature shifting to achieve very high specific and volumetric plasmid yields. This process depends on the preconditioning of the culture with low temperature and restricted growth prior to induction of plasmid amplification in order to reach these very high yields. However, the art teaches that a continuous culture does not have a physiological history; rather, the state of a continuous culture is determined by the dilution rate.

The process of this invention solves this problem by using a first continuous culture stage operated at a reduced temperature to maintain plasmid stability and grow biomass. The culture exiting the first stage enters a second stage with an increased temperature to improve plasmid yield. The residence time of the second stage is limited such that the culture has just enough time to increase its specific plasmid yield without losing plasmid stability. Media may also be added to the second stage to provide substrate for additional biomass growth. The exact temperatures and feed rates can be determined experimentally for each new plasmid by one of average skill in the art. These processes dramatically improve plasmid DNA fermentation productivity, while maintaining plasmid integrity, relative to the processes described in the art. Previously, this novel combination of elements has not been applied to the production of plasmid DNA.

In a preferred embodiment, the temperature in the first stage is in the range of 25-37° C., more preferably in the range of 30-32° C., to produce biomass and maintain plasmid stability. The temperature of the second stage is in the range of 36-45° C. in order to induce plasmid amplification to high plasmid yields. The flow rates of the feed streams entering the first and second stages is determined to allow the culture to stabilize in a mid-log growth phase in the first stage, and to give an optimal residence time for complete induction of plasmid amplification in the second stage. The limiting substrate concentrations entering each stage designed to give the desired biomass yield and to sustain the culture, and these concentrations can be determined by one skilled in the art of continuous culture. In another preferred embodiment this process also includes one or more subsequent stages operated at cooler temperatures to allow completion of plasmid replication and to preserve the culture while harvesting or while waiting to be harvested. In another preferred embodiment the feed rates are controlled to restrict the growth rate between 0.04-0.5 h⁻¹.

In a preferred embodiment, the design equations describing biomass concentration (x₁) and substrate concentration (s₁) in the first stage are the same as those of a single chemostat where the volumetric flow rates into and out of the vessel are maintained constant and equal (F₁):

$$\frac{d}{d t} {x}_{1} ( t ) = \frac{{F}_{1}}{{V}_{1}} \cdot {X}_{1 o} + \frac{{μ}_{max} {r}^{s} {1}^{( t )}}{{K}_{S} + {s}_{1} ( t )} \cdot {x}_{1} ( t ) - \frac{{F}_{1}}{{V}_{1}} \cdot {x}_{1} ( t )$$

$$\frac{d}{d t} {s}_{1} ( t ) = \frac{{F}_{1}}{{V}_{1}} \cdot {S}_{1 o} + \frac{- 1}{{Y}_{xs}} \cdot \frac{{μ}_{max 1} \cdot {s}_{1} ( t )}{{K}_{S} + {s}_{1} ( t )} \cdot {x}_{1} ( t ) - \frac{{F}_{1}}{{V}_{1}} \cdot {s}_{1} ( t )$$

where V₁ is the constant culture volume of the first stage, X₁ₒ is the biomass concentration feed to the first stage (zero in most cases), μₘₐₓ₁ is the maximum specific growth rate of the cells under the conditions of the first stage without nutrient limitation, K_{S} is the Monod saturation constant, Yₓₛ is the yield coefficient of biomass to substrate, and S₁ₒ is the concentration of limiting substrate in the feed to the first stage. The design equations of the second stage are:

$$\frac{d}{d t} {x}_{2} ( t ) = \begin{matrix}\frac{{F}_{1}}{{V}_{2}} \cdot {X}_{1} ( t ) + \frac{{F}_{2}}{{V}_{2}} \cdot {X}_{2 o} + \\ \frac{{μ}_{max 2} {s}_{2} ( t )}{{K}_{S} + {s}_{2} ( t )} \cdot {x}_{2} ( t ) - \frac{{F}_{1} + {F}_{2}}{{V}_{2}} \cdot {x}_{2} ( t )\end{matrix}$$

$$\frac{d}{d t} {s}_{2} ( t ) = \begin{matrix}\frac{{F}_{1}}{{V}_{2}} \cdot {S}_{1} ( t ) + \frac{{F}_{2}}{{V}_{2}} \cdot {S}_{2 o} + \frac{- 1}{{Y}_{xs}} \cdot \\ \frac{{μ}_{max 2} {s}_{2} ( t )}{{K}_{S} + {s}_{2} ( t )} \cdot {x}_{2} ( t ) - \frac{{F}_{1} + {F}_{2}}{{V}_{2}} \cdot {s}_{2} ( t )\end{matrix}$$

where x₂ is the biomass concentration in the second stage, s₂, is the limiting substrate concentration in the second stage, V₂ is the constant culture volume in the second stage, F₂ is the volumetric flow rate of the fresh feed medium into the second stage, S₂ₒ is the concentration of limiting substrate in the fresh feed to the second stage, μₘₐₓ₂ is the maximum specific growth rate of the cells under the conditions of the second stage without nutrient limitation, and X₂ₒ is the biomass concentration in the fresh feed medium into the second stage (equal to zero). The design equations of additional stages may be similar and can be determined by one skilled in the art.

In a preferred embodiment, the duration of the transient startup phase before steady state is achieved is reduced by operating the first stage in batch or fed-batch mode to rapidly accumulate biomass before continuous flow is started.

It is anticipated that the use of various media formulations will result in improved plasmid productivity, using the continuous fermentation processes as described herein. Many media formulations have been described in the art and may be determined by one skilled in the art of microbial cultivation.

### Fermentation Process Alternative Embodiments

In practicing the multistage continuous culture processes of the invention, we contemplate various continuous cultivation strategies including, but not limited to chemostat, turbidostat, feedback control, feed-forward control. Acceptable forms of continuous culture can be determined by those skilled in the art.

In practicing the multistage continuous culture process of the invention, we contemplate various startup procedures for the continuous process. The continuous operation may be started immediately upon inoculation. Alternatively, one or more stages may be started in batch operation to accumulate biomass and reduce the amount of media used before steady state is achieved. Other acceptable methods of startup can be determined by one skilled in the art.

In practicing multistage continuous culture processes of the invention, we contemplate various temperature set points for the different stages.

In practicing the inducible processes with temperature shifting strategies, we contemplate using a final stage with a reduced temperature (in the range of 10-30° C.) to allow plasmid replication to be completed. We have discovered that a cooling period helps to increase plasmid yield and improve plasmid quality (e.g. reduced amounts of nicked and open circle plasmid). We also contemplate a final cooling period to maintain high plasmid quality during downstream purification processes, for example, during alkaline lysis.

In practicing the multistage continuous culture process of the invention, we contemplate sizing the individual volumes of the stages and/or setting flow rates to achieve the desired residence time for each stage. Those skilled in the art will be able to determine acceptable volumes of the stages and flow rates in order to achieve desired residence times.

In practicing multistage continuous culture processes of the invention, we contemplate the use of various types of stages after the first bioreactor stage including, but not limited to bioreactors, fermentors, plug flow reactors, flow through heat exchangers, and continuous stirred tank reactors. Acceptable types and sizes of additional stages can be determined by those skilled in the art.

In practicing the multistage continuous culture process of the invention, we also contemplate the use of bacterial strains that contain inducible autolysis systems along with an additional stage with conditions to induce lysis.

In practicing the multistage continuous culture process of the invention, we also contemplate the use of bacterial strains that contain inducible plasmid-safe nucleases and inducible autolysis systems along with one or more additional stages with conditions to induce lysis and support activity of the plasmid-safe nucleases to degrade contaminating nucleic acids (i.e. host RNA and genomic DNA)

### Plasmids and Host Strains

We contemplate use of the invention in the production of plasmids with a variety of origins of replication, that are either high copy, low copy and moderate copy, and are either temperature inducible or not. Some preferred origins of replication are outlined in Table 1. Modifications to these origins are known in the art, and are also contemplated for use.

TABLE 1

| Parent | | | Copy | |
|---|---|---|---|---|
| Origin | Regulation | High copy Derivation | Number | Therapeutic plasmids |
| pMB1 | Antisense RNAI binds | pUC origin (Rop deletion and second | 50 at 37° C., | Multiple (pcDNA3, |
| | RNAII. Rop accessory | site mutation that alters RNAI/II | 175 at 42° C. | pVAX, VR1012, etc) |
| | protein stabilizes | interaction at 37 and 42, not 30C) | (log phase) | |
| | interaction | pUC origin with second site enhancer | Not | pDNAVACCultra |
| | | increases copy number 14-50% | determined | |
| | | Rop deletion | 30 at 37° C. | pCMVKm2 |
| | | | and 42° C. | |
| | | | (log phase) | |
| | | G to T mutation (extends RNAI, | 1000 (phase | Not described |
| | | attenuating represser; not conditional) | not | |
| | | Plasmid is 65% total DNA | indicated) | |
| ColEI | Same as pMB1 | pMM1, pMM7 (Rop deletion and | 2000 in | pVC0396† |
| | | second site mutation that alters | stationary | |
| | | RNAI/RNAII interaction at 37 and 42, | phase | |
| | | not 30C). pMM7 is >50% total DNA in | (pMM7) | |
| | | stationary phase | | |
| R6K | π rep protein binds | Host strain pir-116 mutant (π rep | 200 | pCpG, pBoost |
| (ori α, | iteron, copy number | protein copy-up mutation in | 250 | pCOR |
| ori β ori | dependent activation | oligomerization domain removed from | | |
| γ) | (low) or repression | plasmid and provided in trans from | | |
| | (high) | chromosome) | | |
| R1 | RepA initiator protein | Temperature inducible copy number | 2000 | Not described |
| | binds non repeated | using dual origin mutant (plasmid is 75% | (mutant) | |
| | target. Antisense CopA | total DNA) | | |
| | represser binds RepA | RepA controlled by temperature | 1000 | pCWH24-6 |
| | leader (CopT). | inducible lambda P_{R} promoter and | (P_{R} | |
| | Auxiliary CopB protein | temperature sensitive lambda repressor | controlled) | |
| | represses RepA | controlled. (Plasmid is 50% total DNA) | | |
| | expression. | | | |
| pKL1 | Rep A initiator protein | Rep A initiator protein overexpression | >2500 | Not described |
| | represses repA | on separate plasmid or on chromosome | | |
| | transcription as | | | |
| | hexamer | | | |

†pVC0396 is an optimized vector backbone, for insertion of eukaryotic expression cassettes

Alternative host strains are contemplated. E. coli strain DH5α is a widely used host for plasmid production. Its key qualities include the recA mutation, which minimizes non-specific recombination of cloned DNA, and the endA1 mutation, eliminating non-specific digestion of plasmid by Endonuclease 1. In addition to DH5α, a variety of other strains are suited for plasmid production; a non limiting list of exemplary E. coli host strains is shown in Table 2.

TABLE 2

| Strain | Genotype | Source |
|---|---|---|
| DH5α | F⁻ Φ80dlacZΔM15 Δ(lacZYA -argF) U169 recA1 endA1 | Invitrogen |
| | hsdR17(rk−, mk+) phoA supE44 λ− thi-1 gyrA96 relA1 | |
| DH10B | F⁻ mcrA Δ(mrr-shdRMS-mrcBC), Φ80dlacZΔM15 ΔlacZ74, | Invitrogen |
| | deoR, recA1, endA1, araD139, Δ(ara-leu)7697, galU, galK, λ−, | |
| | rpsL, nupG | |
| JM109 | endA1, recA1 gyrA96, thi, hsdR17(rk−, mk+) relA1, supE44 | Stratagene |
| | Δ(lac-ProAB) [F′traD36, proAB lacI^{q}ZΔM15] | |
| XL1-Blue | recA1 endA1 gyrA96 thi-1 hsdR17 supE44 relA1 lac [F′proAB | Stratagene |
| | lacI^{q}ZΔM15 Tn10 (Tet¹)] | |
| Top10 | F⁻ mcrA Δ(mrr-hsdRMS-mcrBC) Φ80lacZΔM15 ΔlacX74 | Invitrogen |
| | recA1 araΔ139 Δ(ara-leu)7697 galU galK rpsL (Str^{R}) endA1 | |
| | nupG | |
| Mach1 | ΔrecA1398 endA1 tonA Φ80ΔlacM15 ΔlacX74 hsdR(rₖ⁻mₖ⁺) | Invitrogen |
| GT116 | F⁻ mcrA Δ(mrr-hsdRMS-mcrBC) Φ80lacZΔM15 ΔlacX74 | Invivogen |
| | recA1 endA1 Δdcm ΔsbcC-sbcD | |
| ECOS101 | F⁻ (Φ80 ΔlacZ ΔM15)Δ(lacZYA-argF)U169 hsdR17(r_{κ-}m_{κ+}) | Yeastern |
| | recA1 endA1 relA1 deoR λ− | |
| EC100 | F⁻ mcrA Δ(mrr-hsdRMS-mcrBS) Φ80 ΔlacZ ΔM15)ΔlacX74 | Epicentre |
| | recA1 endA1 araD139(ara, leu)7697, galU, galK, λ− rpsL, | |
| | nupG | |
| Sure | e14− (McrA−) □(mcrCB-hsdSMR-mrr)171 endA1 supE44 thi-1 | Stratagene |
| | gyrA96 relA1 lac recB recJ sbcC umuC::Tn5 (Kan^{r}) uvrC [F′ | |
| | proAB lacI^{q}Z□M15 Tn10 (Tet^{r})] | |
| Stb12 | F⁻ mcrA Δ(mcrBC-hsdRMS-mrr) recA1 endA1 lon gyrA96 thi | Invitrogen |
| | supE44 relAl λ⁻ Δ(lac-proAB) | |
| Stb14 | mcrA Δ(mcrBC-hsdRMS-mrr) recA1 endA1 gyrA96 gal⁻ thi-1 | Invitrogen |
| | supE44 λ⁻ relA1 Δ(lac-proAB)/F′proAB⁺ lacI^{q}ZΔM15 Tn10 | |
| | Tet^{R} | |

DH5α, XL1-Blue, DH10B, JM109 and Top 10 have been well established as plasmid production strains. Mach1 and ECOS101 have been developed recently and may be desirable plasmid production hosts. Stb12, GT116 and Sure cells have been utilized for the production of unstable DNA containing plasmids. Unstable DNA contains structures like direct (e.g. retroviral long terminal repeats) or inverted repeats (e.g. shRNA palindromes), Z DNA, etc. The deletion of the dcm gene in GT116 eliminates dam methylation which is immuno-stimulatory. Therefore, production in GT116 reduces the immunogenicity of plasmid DNA. Similar reductions in immunogenicity are observed utilizing strains expressing CpG methylase.

### Production of Unstable Plasmids

We also contemplate use of the invention in the production of plasmids containing unstable sequences. Palindrome sequences, direct or inverted repeats, and Z DNA forming sequences are unstable and are deleted or rearranged by E. coli hosts. In some instances, plasmids for therapeutic use must contain unstable sequences (inverted or direct repeats for viral vectors such as AAV and HIV, Z DNA forming segments or triplet repeats for certain therapeutic genes). Current strategies to maintain plasmids containing unstable sequences are to use host cell lines with stabilizing mutations. Several hosts are commercially available for propagation of these plasmids, for example, Sure cells (Stratagene), GT115 (Invivogen) or Stb12 and Stb14 (Invitrogen). The Stb12 and Stb14 cell lines utilize an undisclosed mutation that increases stability of direct repeat containing vectors such as retroviral vectors; this effect is enhanced at reduced temperature, presumably due to reduced copy number. Specific combinations of repair mutations can stabilize plasmid propagation, especially at low temperature. The Sure and Sure2 cell lines uses one such combination, with homologous recombination deficiency (recB, recJ) in conjunction with UV repair (uvrC) and SOS repair (umuC) deficiency (to stabilize LTRs), and SbcC (and recJ) to stabilize Z DNA. The GT116 cell line uses SbcC and SbcD to stabilize palindromic sequences. These strains function to stabilize plasmids only at low temperature (i.e. 30° C.), presumably due to reduced plasmid copy number. This strategy obviously increases production cost. Use of the inducible fermentation process described herein would allow propagation at 30° C. of unstable plasmids in stabilizing cell lines, prior to increasing copy number only for a short duration prior to harvest. This should maximize yield and stability (i.e. quality) of unstable plasmids.

### Improved Host Strains for Plasmid Production

### DNA Compaction and Plasmid Production

The underlying mechanism for the observed increased yield of plasmid DNA (>6 mg/L/OD₆₀₀) in the inducible fed-batch process is unknown. It is potentially due to induction of DNA compaction agents (e.g. histone like protein or other chromatin binding proteins, such as the dps gene product) during biomass production at slow growth and reduced temperature.

Altering DNA condensation during the induction phase may increase plasmid yield by increasing tolerable plasmid levels or copy number. The degree of compaction of a DNA is set by two opposing factors; condensing chromatin proteins and decondensing transcription complexes. Plasmid compaction may be affected by the level of transcription from plasmid promoters. Less transcription may be associated with higher compaction, and potentially higher carrying capacity.

In E. coli, a number of chromatin proteins have been identified that are involved in DNA compaction. These gene products bind plasmid and genomic DNA. In the case of genomic DNA, they compact the DNA into the nucleoid (reviewed in: Robinow & Kellenberger, Microbiol. Rev. 58:211-232, (1994)). The major components of the nucleoid are the histone like proteins HU, IHF, and HN-S, StpA (related to HN-S, expressed at about 1/10 level) and Dps, which are distributed uniformly in the nucleoid, while other proteins, such as SeqA, CbpA, CbpB, Fis and IciA are in lower amount, show non-uniform distribution in the nucleoid, and may have regulatory functions. An isolated R plasmid protein complex contained three major proteins, 23% HN-S, 23% RNA polymerase, and 5% HU. This would presumably change depending on growth phase since chromatin associated gene products are differentially regulated in different media, different cell densities and during growth and stationary phases. F is, HU, HF-1 generally are more highly expressed in log phase, while IHF and Dps are at higher levels in stationary phase. Dps compacts DNA into highly dense liquid biocrystalline complexes in stationary phase to improve stress resistance. Over-expression of HN-S during the growth phase leads to DNA condensation and viability loss. Cells may have higher plasmid capacity when DNA is highly compacted. In the NTC inducible fermentation process, growth phase cells have lower overall capacity for plasmid DNA than induction phase cells. This may be due to differences in the combinations of chromatin proteins present in the induction phase which may allow higher levels of tolerable plasmid than in the growth phase. Alternations to the ratios of chromatin proteins during the induction phase may increase plasmid compaction, and carrying capacity.

Alternations to the ratios of chromatin proteins during the induction phase may also increase plasmid replication rates. For example, expression from the p15A origin RNAII promoter, but not the pMB1 (pBR322) RNAII promoter, is repressed by IHF; p15A RNAII transcription is increased in IHF mutants. Dps and HU are non-specific DNA binders, HN-S, CbpA and CbpB bind curved DNA. StpA is related to HN-S, binds DNA with higher affinity, and also binds curved DNA. F is, IHF, IciA and seqA are sequence specific. HN-S represses transcription from a number of promoters that contain curved DNA. The RNAII promoter of pMB1 (pUC and pBR322) contains polyA and poly T tracks; these sequences form curved DNA. The decreased levels of curved DNA binding chromatin proteins that repress transcription (e.g. HN-S) in stationary phase may be associated with increased ratio of RNAII to RNAI transcription, and the documented stationary phase increase in plasmid copy number. Alterations to the composition of the chromatin proteins (e.g. further decrease in HN-S) present during the production phase of NTC fermentation process may lead to increased plasmid copy number with pMB1 plasmids such as pUC.

Heterologous DNA compactors, for example, the acid soluble spore proteins of Bacillus species, when expressed in E. coli, may also be useful DNA compactors for increasing plasmid yield. For example, expression of a B. subtilis small acid soluble protein in E. coli causes DNA compaction (Setlow et al., J. Bact., 173:1642-1653, (1991).

Process alterations may also improve yields through effects on DNA condensation. Dps compaction is regulated by Magnesium (Mg⁺⁺) concentration; the presence of Dps does not result in DNA condensation; tightly packed crystalline DNA:Dps complexes form when Mg⁺⁺ concentration falls below a threshold (reviewed in: Frenkiel-Krispin et al., EMBO J. 20:1184-1191, (2001)). Morphologically, the complex resembles that induced by chloramphenicol addition during stationary phase. Addition of 0.2 mM spermidine to growing cultures accelerates DNA condensation in the absence of Dps. Phosphate starvation has the same effect, perhaps through enhanced degradation of threonine and arginine to spermidine (Frenkiel-Krispin et al, Supra). Changes to fermentation composition or conditions during the induction phase, to alter the levels of divalent cations (e.g. Mg⁺⁺, through exogenous addition or depletion), or alter the level of positively charged polyamines (e.g. spermidine, through exogenous addition or control of bacterial synthesis) may increase plasmid yield.

We contemplate further yield increases may be obtained by further compaction of plasmid DNA. This could be achieved by addition of DNA compaction agents to the feed (e.g. polyethyleneimine, spermidine, spermine) or strain modifications that increase production of host strain DNA compaction agents such as spermine production or dps protein production, during the fermentation process. Such strain modifications could be alterations that allow the relevant gene products to be induced during the fermentation process.

In practicing the continuous culture processes, we contemplate using alternative strategies to maintain plasmid copy number at a low level in the growth stage. For example, in addition to growth at a low temperature, other mechanisms exist to reduce copy number that e incorporated into the growth phase. For example, reduced dissolved oxygen during fermentation has been shown to reduce plasmid copy number (see Carnes, BioProcess International 9:36-42, (2005).

### Improvement to Final Product Purity

We contemplate utilizing plasmid enriched feed streams from the described fermentation culture in exemplary plasmid purification processes. Such processes are well known in the art. The combination of high yield fermentation and exemplary purification process may provide cost effective methodologies to further reduce genomic DNA levels to acceptable levels for gene therapy and DNA vaccination applications.

### EXAMPLES

The method of the invention is further illustrated in the following examples. These are provided by way of illustration and are not intended in any way to limit the scope of the invention.

### Example 1

### Inducible Fed-Batch Process for High Yield Production of High Copy Plasmids with NTC3019 Media

The plasmid gWiz GFP in DH5α was utilized in a inducible fed-batch process. NTC3019 fed-batch fermentation was grown at 30° C. until 60 OD₆₀₀, at which time the temperature was shifted to 37° C. The surprising results are shown in FIG. 1A. Growth at 30° C. through 60 OD₆₀₀ eliminated the growth arrest problem, and the culture ultimately exceeded 100 OD₆₀₀ with a total plasmid yield of 670 mg/L. The DNA purified from samples from this process is of a high quality, being essentially 100% supercoiled with no detectable deletion or other rearrangement.

Plasmid yields prior to the temperature shift remained low throughout the growth phase, remaining below 2 mg/L/OD₆₀₀. This is in contrast to the results from 33° C. or 37° C. fermentations. Remarkably, the specific plasmid yields after temperature shift are very high, up to 6.5 mg/L/OD₆₀₀, well exceeding levels observed with other fermentation media/processes. Fermentation at 30° C. through the growth phase, and shifting to 42° C. resulted in productivity yields of 1.1 gm/L (11 mg/L/OD₆₀₀) after 42 hours with gWiz-GFP (FIG. 1B). Productivity plateau is not associated with extensive cell death, as the majority of the cells remain viable.

Modification of the NTC3019 media (four fold increase in glycerol, yeast extract, and magnesium in the batched media) to reduce the duration of the fed-batch phase (by extending the batch phase to higher OD₆₀₀) also produced similarly high plasmid yields after induction at 42° C., demonstrating that the fed-batch phase can be started at higher OD₆₀₀ without loss of plasmid induction.

Multiple different plasmids with various pUC origin backbones, including different antibiotic resistance genes and orientations of prokaryotic elements, have been produced in yields greater than 0.5 g/L in NTC3019 media, using the 30° C. to 42° C. inducible process in DH5α. These results demonstrate that the inducible process is not specific to a particular plasmid.

The gWiz-GFP plasmid was also produced in yields greater than 500 mg/L in NTC3019 media, using the 30° C. to 42° C. inducible process, in the DH1 cell line. This result demonstrates that the inducible process is not specific to a particular E. coli strain.

As well, DNA purifications at the 1 gram scale have been performed utilizing the cells from this process. This demonstrates inducible fermentations performed in NTC3019 fed-batch media are amenable to large scale downstream processing.

Expressing plasmid yields in terms of specific yields (mg/L/OD₆₀₀) indicates the amount of plasmid relative to the total cell mass. The inducible fed-batch process described herein maintained low (<2 mg/L/OD₆₀₀) plasmid levels throughout the biomass phase of the process, and facilitated unprecedented ultra high plasmid production (6-11 mg/L/OD₆₀₀) after biomass production. High specific yields are very desirable since increased plasmid yield per gram of bacteria leads directly to higher final product purities.

### Example 2

### Two Stage Continuous Culture

Plasmid gWiz GFP in E. coli DH5α was inoculated into the Stage 1 bioreactor at T₁=30° C., with 2 L of medium containing, per liter:

| Component | |
|---|---|
| | grams |
| Yeast extract | 14 |
| Potassium phosphate monobasic, KH₂PO₄ | 2.4 |
| Sodium chloride, NaCl | 0.5 |
| Sodium phosphate dibasic anhydrous, Na₂HPO₄ | 6 |
| Citric Acid anhydrous | 1.5 |
| Nanopure Water | 60 |
| Magnesium sulfate heptahydrate, MgSO₄•7H₂O | 3.2 |
| Glycerol | 60 |
| | ml |
| Kanamycin 50 mg/ml stock solution | 1 |
| Thiamin HCl 0.5% stock solution | 1 |
| Trace Minerals Solution | 10 |

The starting OD₆₀₀ in Stage 1 was 0.01. This was grown in batch mode until 17:20 hours post inoculation. At this point the Stage 1 culture was at OD₆₀₀ 25.8. Feed and effluent flows for Stage 1 were started at this time. An illustration of the multistage bioreactor system in this example is shown in FIG. 2.

The composition of feed medium into Stage 1 was the same as the starting medium, shown above. The substrate (glycerol) concentration in the Stage 1 feed, S₁ₒ, was 60 g/L.

The feed and effluent flow rates for Stage 1 were equal to maintain constant volume, F₁=0.24 L/h. The effluent from Stage 1 was removed by means of an overflow dip tube. The specific plasmid yield in Stage 1 remained low over the duration of the experiment, as expected, between 1.1 and 3.3 mg/L/OD₆₀₀.

The effluent from Stage 1 was pumped into Stage 2, initially containing 8 L of sterile medium with the same composition as shown above. The temperature of Stage 2, T₂, was 42° C.

Stage 2 had its own nutrient feed to provide nutrients during plasmid accumulation. Stage 2 feed was pumped in at F₂=0.08 L/h, and had the following composition, per liter:

| Component | |
|---|---|
| | grams |
| Yeast extract | 4 |
| Potassium phosphate monobasic, KH₂PO₄ | 2.4 |
| Sodium chloride, NaCl | 0.5 |
| Sodium phosphate dibasic anhydrous, Na₂HPO₄ | 6 |
| Citric Acid anhydrous | 1.5 |
| Nanopure Water | 60 |
| Magnesium sulfate heptahydrate, MgSO₄•7H₂O | 3.2 |
| Glycerol | 250 |
| | ml |
| Kanamycin 50 mg/ml stock solution | 1 |
| Thiamin HCl 0.5% stock solution | 1 |
| Trace Minerals Solution | 10 |

Effluent from Stage 2 was removed by an overflow dip tube at a rate of F₁+F₂=0.32 L/h to maintain a constant volume in Stage 2. One skilled in the art will realize that volume additions due to acid and base for pH control are also removed in the effluent of both stages and can be considered negligible.

After 64 hours of continuous operation the specific plasmid yield in Stage 2 stabilized at 12 mg/L/OD600 and the cell density of Stage 2 was OD600 66, resulting in a continuous production stream containing approximately 800 mg/L plasmid DNA at 0.32 L/h. Agarose gel electrophoresis of plasmid DNA isolated from samples from each stage over show that the plasmid was stable in both stages over a range of continuous operation (FIG. 3.).

### Example 3

### Two Stage Continuous Culture for High Yield Production of a Gene Therapy Plasmid

One skilled in the art will recognize that the flow rates and residence times for each stage can be optimized for maximum yield and productivity. Based on the results in Example 2, the productivity may be improved by adjusting feed rates and residence times for each stage. A temperature inducible plasmid may be produced by the generic process illustrated in FIG. 4.

By way of example, a method for continuous production of DH5α/gWiz-GFP is discussed below. Vessel 1 contains an initial 8.0 L volume of batch medium at 30° C. After inoculation with plasmid-containing E. coli, the culture in Vessel 1 is allowed to reach 20 g dry cell weight per liter (X₁=20 g/L, mid-log growth) in batch mode.

Continuous culture is performed with a temperature of 30° C. in Vessel 1 and 42° C. in Vessel 2. The media volume in Vessel 2 is 10 L. For example, the specific plasmid yield of gWizGFP in DH5α will rise to 12 mg/L/OD₆₀₀. Previous experiments have shown that the specific plasmid yield will reach this level after 7-9 hours at 42° C. Thus, this process is operated to give a residence time in Vessel 2 of 8.6 hours.

This system is illustrated by FIG. 4, having the following values: F₁=0.96 L/h, X₁ₒ=0 g/L, S₁ₒ=60 g/L, T₁=30° C., F₂=0.2 L/h, X₂ₒ=0 g/L, S₂ₒ=400 g/L, T₂=42° C. The yield coefficient from glycerol, the limiting substrate, is 0.4 g dry cell weigh per g glycerol. The maximum specific growth rate, μₘₐₓ₁, of E. coli DH5α/gWizGFP at 30° C. is 0.26 h⁻¹. The maximum specific growth rate, μₘₐₓ₂, of E. coli DH5α/gWizGFP at 42° C. is about 0.07 h⁻¹. μₘₐₓ₂ is lower due to the increased metabolic load caused by the high plasmid production at 42° C.

Vessel 1 is initially operated in batch mode to accumulate an initial biomass concentration of X₁=20 g/L dry cell weight for the start of continuous operation. FIG. 5. displays the graphical results of this two stage continuous culture process beginning at time 0 hours with an initial biomass concentration of X₁=20 g/L dry cell weight. The horizontal axis has units of hours and the vertical axis has units of grams per liter.

After steady state is achieved Vessel 1 has 24 g dry cell weight (DCW) per liter, or an OD₆₀₀ of about 44, with a specific plasmid yield of 1.2 mg/L/OD₆₀₀; overall plasmid yield leaving Vessel 1 is 52 mg/L. The effluent stream of Vessel 2 has a flow rate of 1.16 liters per hour, a biomass concentration of 46 g DCW/L, or OD₆₀₀ of about 85, and a specific plasmid concentration of 12 mg/L/OD₆₀₀. The resulting continuous stream exiting Vessel 2 has a plasmid yield of 1020 mg/L. Overall plasmid productivity is 1.2 grams of plasmid DNA per hour.

### Example 4

### Further Plasmid Yield Increases and High Plasmid Quality Through Use of Reduced Temperatures at the Final Stage of Cultivation

A DNA vaccine plasmid in E. coli DH5α was produced using the inducible fed-batch process shown in FIG. 6. After the culture reached OD₆₀₀ 75, the plasmid yield was 663 mg/L. The culture was cooled to between 15° C. and 25° C. for 30 minutes, over which period there was no increase in biomass. After this cooling period the plasmid yield had risen 29% to 814 mg/L. This increase is possibly due to completion of all plasmid replication that was still ongoing at the higher temperatures.

We contemplate the use of a cooling stage after the two stage continuous culture process to further increase plasmid yield and enhance plasmid quality. This may be an additional fermentation vessel or another type of temperature controlled vessel.

### CONCLUSIONS, RAMIFICATIONS AND SCOPE OF THE INVENTION

Thus, the reader will see that the associated production processes of the invention provide compositions and methods for improved plasmid production.

While the above description contains many specificities, these should not be construed as limitations on the invention, but rather as an exemplification of one preferred embodiment thereof. Many other variations are possible.

I claim:

1) A method for continuous production of covalently closed super-coiled plasmid DNA comprising the steps of:
a. growing microbial cells containing a plasmid, cosmid, or bacterial artificial chromosome replicon at a reduced temperature in a first continuous culture stage; and
b. inducing plasmid DNA production by directing the effluent of the continuous culture stage in part (a) into a second plasmid induction continuous culture stage with an increased temperature; and
c. operating the plasmid induction continuous culture stage with a residence time that allows accumulation of plasmid product; and
d. continuously harvesting cells from the second continuous culture stage;
whereby said method enables continuous production of microbial cells containing plasmid DNA.

2. The method of claim 1 wherein the reduced temperature during the first continuous culture is approximately 30° C.

3. The method of claim 1 wherein the increased temperature in the second continuous culture stage is in the range of 36-45° C.

4. The method of claim 1 wherein said harvested cells are held at a cooler temperature to increase final plasmid yield.

5. The method of claim 1 wherein said harvested cells are E. coli cells.

6. A method comprising holding plasmid-containing microbial cells after cell growth at a cooler temperature in order to increase final plasmid yield.
